(19) United States
(12) Patent Application Publication
(10) Pub. No.: US 2009/0291057 A1
(43) Pub. Date: Nov. 26, 2009

(54) ANIMAL CARE COMPOSITION

(76) Inventors: Debora W Chang, Mason, OH (US); Shellie Jean Porter Caudill, Liberty Township, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/466,409

(22) Filed: May 15, 2009

Related U.S. Application Data
(60) Provisional application No. 61/054,834, filed on May 21, 2008.

(51) Int. Cl. A61K 8/30; A61Q 5/12

(52) U.S. Cl. 424/70.21; 424/70.1

(57) ABSTRACT

Methods of cleansing, conditioning and optionally therapeutically treating skin, hair, and/or other keratinous surfaces of a companion animal are provided. The method includes: contacting the skin, hair, and/or other keratinous surfaces of a companion animal with a companion animal care composition, the composition having a cleansing component, and a conditioning component; and optionally rinsing the skin, hair, and/or other keratinous surfaces of the companion animal with water; wherein after use of the composition, the composition provides a friction coefficient of from about 0.65 to about 0.8. Also included are articles of commerce having a container containing a companion animal care composition, which composition provides cleansing, conditioning, and optional treatment benefits to a companion animal. The container has instructions for cleansing, conditioning, and optionally treating the companion animal. Also included are companion animal care compositions having a cleansing component and a conditioning component, wherein after use of the composition, the composition provides a friction coefficient of from about 0.65 to about 0.8.

### CROSS REFERENCE TO RELATED APPLICATION

This application claims the benefit of U.S. Provisional Application No. 61/054,834, filed May 21, 2008.

### FIELD OF THE INVENTION

The present invention relates to companion animal care compositions. More particularly, the present invention relates to companion animal care compositions, methods, systems, and articles of commerce that provide cleansing, conditioning, and optionally treatment of skin, hair and other keratinous surfaces of a companion animal.

### BACKGROUND OF THE INVENTION

Various types of cleansing compositions are known for use with domestic and companion animals, such as cats, dogs, horses, and the like. Compositions for the care and grooming of animals often require special components that are particularly effective for providing certain benefits and treatments such as treating the skin of the animal, dandruff reduction, providing sheen and luster to the coat, moisturizing, allergy reduction, antibacterial function, odor control, flea, tick and mosquito control, fragrance, and various combinations thereof. However, such compositions must also be pleasant and non-toxic to the animal. The compositions must have appropriate skin feel, mildness to skin, hair, and ocular mucosae, as well as a pleasant smell and appropriate lather volume. Such compositions can also be edible because most animals groom themselves to some extent and therefore are likely to ingest a portion of any composition applied to the animal's body and/or coat.

Such compositions are typically a shampoo which is typically an aqueous solution, emulsion or emersion of one or more cleansing agents, with optional actives for providing, modifying, and/or stabilizing various functions and properties of the composition. Such additives include conditioning agents, solubilizers, foam modifying agents, dispersants, medicaments, pH controlling agents, preservative systems, fragrances, and the like.

Such compositions are typically generally in thick, viscous, liquid form and are contained in and dispensed from a container such as a plastic bottle having a screw-on cap. Generally, to bathe an animal, a user must pre-wet the animal's coat, apply the cleansing composition, work the composition into a lather, and then rinse, dry and/or groom the animal. However, such dispensing containers can be awkward to handle with one hand, while attempting to hold a wet animal. Therefore, pump action dispensers for animal care compositions have been developed to supply mist and/or foam. Compositions for use in such containers and dispensers, with appropriately adjusted viscosity and foaming action, have also been developed. Compositions that rinse more easily and thoroughly than traditional shampoos have also been developed.

In addition, there exist various “waterless” and/or “leave-on” animal bathing compositions that are pre-applied to a cloth type material and can simply be wiped over the animal. Other waterless and/or leave-on compositions include foam type shampoo compositions that can be dispensed onto the animal's coat and left thereon. The compositions are dispensed onto the coat, worked into the coat, and the animal is dried with no need to pre-wet or rinse the animal. Such compositions were developed in part to address the physical difficulties in bathing an animal that does not like or want to be bathed. Such compositions are easier for the bather/owner/handler to handle and apply to the animal, and should make the bathing experience more pleasant and less traumatic to the animal.

Furthermore, such compositions are desirable because compositions that require a water source to remove the cleansing composition might not be completely removed in the rinsing process. If the composition is not completely removed, the animal may show signs of irritation such as itching, which can result in a ‘hot spot’ being formed on the animal's skin. Additionally, locating an appropriate water source can be inconvenient or impossible under certain conditions due to travel, time constraints and/or size of the animal. Such waterless and/or leave-on compositions must be non-irritating to the skin and mucus membranes, and must be non-toxic to the animal because such compositions will likely be ingested to some degree.

There also exist shampoo compositions with extended fragrance such that the animal smells clean and pleasant to the owner for a longer period of time. In addition, there are shampoos for animals of different species, skin type, coat type, and size. Some examples of such composition include Sleeky® conditioning pet shampoo for silky, thick soft hair; Sleeky® flea & tick conditioning shampoo for short hair; Friskies® neutral pH shampoo (non-irritating); Friskies® Pro-Control dog shampoo conditioning shampoo for long haired dogs; Gold Medal long hair pet shampoo; Mikki softening dog shampoo for coarse or wiry hair coats; Miracle Coat® spray-on dog shampoo; Miracle Coat® spray on puppy shampoo; OxyPet shampoo and conditioner mousse with a built-in brush; and various aerosol products.

However, most such products are generally a shampoo product. Shampoo products can be drying to the skin and coat. Therefore, there is often a need for a separate conditioner to be used. However, using a traditional separate shampoo and conditioner can add to the stress level of both the animal and the person bathing the animal.

Therefore, there remains a need for animal care compositions and methods that provide cleansing and conditioning, and optionally treatment appropriate for different species of animal, different breeds of animal, different coat types, different skin types, and different medical conditions. There also remains a need for animal care compositions that are easy to handle and use for the owner/handler; which provide appropriate cleansing, conditioning, and optionally treatment for the animal while being mild and non-irritating to the animal's skin and oral mucosae, and non-toxic if ingested; and which improve the bathing experience for the animal being bathed and the owner/handler bathing the animal.

### SUMMARY OF THE INVENTION

The present invention is a companion animal care composition having a cleansing component and a conditioning component, wherein after use of the composition, the composition provides a friction coefficient of from about 0.65 to about 0.8. Also included in the present invention are methods of cleansing, conditioning, and optionally therapeutically treating skin, hair, and other keratinous surfaces of a companion animal; the method having the steps of: contacting the skin, hair, and other keratinous surfaces of a companion animal with a companion animal care composition; the composition having a cleansing component, a conditioning component; and rinsing the skin, hair, and other keratinous surfaces of the companion animal with water; wherein after use of the composition, the composition provides a friction coefficient of from about 0.65 to about 0.8. Also included are articles of commerce having a container containing a companion animal care composition, which composition provides cleansing, conditioning, and optionally treatment benefits to a companion animal when applied to the companion animal. The container has instructions for cleansing, conditioning, and optionally treating the companion animal. The invention also includes a companion animal care system comprising an array of the companion animal care compositions, each composition comprising the array being tailored for use with a particular coat type.

### DETAILED DESCRIPTION OF THE INVENTION

The present invention is a companion animal care composition having a cleansing component and a conditioning component, wherein after use of the composition, the composition provides a friction coefficient of from about 0.65 to about 0.8. The compositions of the present invention provide for methods of cleansing, conditioning, and optionally therapeutically treating skin, hair, and other keratinous surfaces of a companion animal. The compositions of the present invention can also be provided in articles of commerce having a container containing the composition, and instructions for use of the composition. The compositions can also form a companion animal care system having an array of compositions, each composition tailored for use with a particular coat type, and indicia for selection of the appropriate composition.

As used herein, the term “companion animal” includes dogs; cats; horses; rabbits; guinea pigs; hamsters; gerbils; ferrets; farm animals including cattle, goats, pigs, llamas, alpacas; zoo mammals; and the like. Dogs, cats, horses and rabbits are particularly applicable.

As used herein the term “therapeutic” if used before the term “treatment” and use of the term “treatment” alone includes preventive and curative treatment, e.g. treatment to prevent biting and infestation by fleas and/or ticks, and treatment to kill and eliminate existing flea and/or tick populations.

As used herein, all examples and groups of examples of compounds and/or components suitable for use in the present invention are non-limiting examples thereof, regardless of whether stated explicitly as non-limiting examples.

All examples and groups of examples of compounds and/or components suitable for use in the present invention can also be mixtures thereof, regardless of whether mixtures are stated explicitly.

### Coat Types and Needs

While it is possible to differentiate companion animal coat types by hair length, hair texture and/or hair thickness, the potential permutations of these three characteristics quickly becomes exhaustive. Thus, broader coat type categories can be defined to be sufficiently inclusive to encompass the majority of coat types of species and breeds commonly owned as companion animals.

For ease of discussion herein, non-limiting examples of common canine coat type descriptors are: short and smooth, long and silky, fluffy, and wiry, and include those coat types known to those of skill in the art as being “fur” and those known as being “hair”. However, it is understood that there can be other terms used to describe various canine coat characteristics. Non-limiting examples of canine breeds with short and smooth coats include Labrador Retriever, Beagle, Smooth Dachshund, Chihuahua, and Rottweiler. Non-limiting examples of canine breeds with long and silky coats include Afghan Hound, Border Collie, Irish Setter, Maltese, and Cocker Spaniel. Non-limiting examples of canine breeds with fluffy coats include Pomeranian, Chow Chow, Poodle, Bichon Frise, and American Eskimo Dog. Non-limiting examples of canine breeds with wiry coats include Cairn Terrier, Standard Schnauzer, German Wirehaired Pointer, Airedale Terrier, and Welsh Terrier.

While short and smooth coats do not tangle or mat, owners of companion animals of this type still desire the coat to be shiny, smooth, and silky but not oily. Consequently short and smooth coats call for an animal care composition that provides low to moderate levels of conditioning.

It has been found that long and silky coats have a tendency towards tangling, matting, and hair breakage. Owners of long and silky coated companion animals desire the coat to be soft, silky, and highly conditioned to prevent hair breakage and allow for the easy removal of tangles and mats. Therefore long and silky coats call for an animal care composition that provides high levels of conditioning.

Fluffy coats have a tendency towards matting as well as frizzing. Owners of companion animals of this type desire the coat to be soft, full of volume, and not weighed down, but still well conditioned to allow for easy removal of mats. Fluffy coats call for an animal care composition with moderate levels of conditioning.

Wiry coats tend to be coarse, rough, and mat readily. Owners of wiry coated companion animals desire the coat to be soft and well conditioned to allow for the easy removal of mats. Therefore wiry coats call for an animal care composition that provides moderate levels of conditioning. The compositions, methods, and articles of commerce of the present invention provide both cleansing and appropriate conditioning for different coat types, as well as provide optional therapeutic and/or preventive treatment components and methods.

### Compositions

### I. Cleansing Component

The cleansing component of the present invention, in most general terms, can include anionic surfactant, non-ionic surfactant, amphoteric surfactant, zwitterionic surfactant, cationic surfactant, and mixtures thereof, which are described in detail herein.

### Surfactant

The compositions of the present invention can include a detersive surfactant. The detersive surfactant component is included to provide cleansing performance to the composition. The detersive surfactant component in turn comprises anionic detersive surfactant, zwitterionic and/or amphoteric detersive surfactant, as well as non-ionic surfactant and cationic surfactant, and/or a mixture thereof. Such surfactants should be physically and chemically compatible with the essential components described herein, and should not otherwise unduly impair product stability, aesthetics, or performance.

Anionic Surfactant Suitable anionic detersive surfactant components for use in the composition herein include those which are known generally for use in human hair care or other personal care cleansing compositions. The concentration of the anionic surfactant component in the composition should be sufficient to provide the desired cleaning and lather performance. The anionic surfactant is present at a concentration of from about 5% to about 50%, alternatively from about 8% to about 30%, alternatively from about 10% to about 25%, and alternatively from about 12% to about 22%, by weight of the composition.

Generally, non-limiting examples of suitable anionic surfactants include sarcosinates, sulfates, sulfonates, isethionates, phosphates, taurates, lactylates, glutamates, carboxylates, and mixtures thereof.

Non-limiting examples of anionic surfactants particularly suitable for use in the compositions are alkyl and alkyl ether sulfates. These materials have the respective formulae ROSO₃M and RO(C₂H₄O)ₓSO₃M, wherein R is alkyl or alkenyl of from about 8 to about 18 carbon atoms, x is an integer having a value of from 1 to 10, and M is a cation such as ammonium, alkanolamines such as triethanolamine, monovalent metals such as sodium and potassium, and polyvalent metal cations such as magnesium, and calcium.

R has from about 8 to about 18 carbon atoms, alternatively from about 10 to about 16 carbon atoms, and alternatively from about 12 to about 14 carbon atoms, in both the alkyl and alkyl ether sulfates. Alkyl ether sulfates can be made as condensation products of ethylene oxide and monohydric alcohols having from about 8 to about 24 carbon atoms. The alcohols can be synthetic or they can be derived from fats, e.g., coconut oil, palm kernel oil, tallow, and mixtures thereof. Lauryl alcohol and straight chain alcohols derived from coconut oil or palm kernel oil are particularly suitable. Such alcohols are reacted with from about 0 to about 10, alternatively from about 2 to about 5, and alternatively about 3, molar proportions of ethylene oxide. The resulting mixture of molecular species having, for example, an average of about 3 moles of ethylene oxide per mole of alcohol, is sulfated and neutralized.

Non-limiting examples of other suitable anionic detersive surfactants include water-soluble salts of organic, sulfuric acid reaction products conforming to the formula [R¹—SO₃-M] where R¹ is a straight or branched chain, saturated, aliphatic hydrocarbon radical having from about 8 to about 24, and alternatively from about 10 to about 18 carbon atoms; and M is a cation described hereinbefore.

Non-limiting examples of still other suitable anionic detersive surfactants include reaction products of fatty acids esterified with isethionic acid and neutralized with sodium hydroxide where, for example, the fatty acids are derived from coconut oil or palm kernel oil; sodium or potassium salts of fatty acid amides of methyl tauride in which the fatty acids, for example, are derived from coconut oil or palm kernel oil, and mixtures thereof. Other similar anionic surfactants are described in U.S. Pat. Nos. 2,486,921; 2,486,922; and 2,396,278.

Non-limiting examples of other anionic detersive surfactants suitable for use in the compositions include succinnates, non-limiting examples of which include disodium N-octadecylsulfosuccinnate; disodium lauryl sulfosuccinate; diammonium lauryl sulfosuccinate; tetrasodium N-(1,2-dicarboxyethyl)-N-octadecylsulfosuccinnate; diamyl ester of sodium sulfosuccinic acid; dihexyl ester of sodium sulfosuccinic acid; dioctyl esters of sodium sulfosuccinic acid; and mixtures thereof.

Non-limiting examples of other suitable anionic detersive surfactants include olefin sulfonates having from about 10 to about 24 carbon atoms. In addition to the true alkene sulfonates and a proportion of hydroxy-alkanesulfonates, the olefin sulfonates can contain minor amounts of other materials, such as alkene disulfonates depending upon the reaction conditions, proportion of reactants, the nature of the starting olefins, impurities in the olefin stock, and side reactions during the sulfonation process. A non-limiting example of such an alpha-olefin sulfonate mixture is described in U.S. Pat. No. 3,332,880.

Another non-limiting class of anionic detersive surfactants suitable for use in the compositions is the beta-alkyloxy alkane sulfonates. These surfactants conform to the Formula (I):

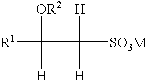

where R¹ is a straight chain alkyl group having from about 6 to about 20 carbon atoms, R² is a lower alkyl group having from about 1 to about 3 carbon atoms, alternatively 1 carbon atom, and M is a cation described hereinbefore.

Non-limiting examples of suitable anionic detersive surfactants for use in the compositions include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate, and mixtures thereof.

### Amphoteric and Zwitterionic Surfactants

Non-limiting examples of suitable amphoteric or zwitterionic detersive surfactants for use in the composition herein include those which are generally known for use in human hair care or other personal cleansing compositions. As used herein, the term “amphoteric surfactant” can encompass zwitterionic surfactants which are well known to formulators skilled in the art as a subset of amphoteric surfactants. Concentration of such amphoteric or zwitterionic detersive surfactants ranges from about 0.5% to about 20%, and alternatively from about 1% to about 10%. Non-limiting examples of suitable zwitterionic or amphoteric surfactants are described in U.S. Pat. Nos. 5,104,646; and 5,106,609.

Amphoteric detersive surfactants suitable for use in the composition are well known in the art, and include those surfactants broadly described as derivatives of aliphatic secondary and tertiary amines in which the aliphatic radical can be straight or branched chain and wherein one of the aliphatic substituents contains from about 8 to about 22 carbon atoms, and one contains an anionic group such as carboxy, sulfonate, sulfate, phosphate, or phosphonate. Generally, non-limiting examples of suitable amphoteric detersive surfactants include alkyliminoacetates, iminodialkanoates, aminoalkanoates, and derivatives and mixtures thereof. As used herein, the term “derivative” is used as it would generally understood by one of skill in the art, i.e. to mean a compound that is formed from a similar compound, or a compound that can arise from another compound, if one atom is replaced with another atom or group of atoms. Non-limiting examples of amphoteric detersive surfactants particularly suitable for use in the present invention include cocoamphoacetate, cocoamphodiacetate, lauroamphoacetate, lauroamphodiacetate, and mixtures thereof.

Zwitterionic detersive surfactants suitable for use in the composition are well known in the art, and include those surfactants broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group such as carboxy, sulfonate, sulfate, phosphate or phosphonate. Generally, suitable zwitterionic detersive surfactants include betaines, sultaines and hydroxysultaines. Non-limiting examples of zwitterionic surfactants particularly suitable for use in the present invention are betaines. Examples of betaines include higher alkyl betaines such as coco dimethyl carboxymethyl betaine, lauryl dimethyl carboxymethyl betaine, lauryl dimethyl alpha-carboxyethyl betaine, cetyl dimethyl carboxymethyl betaine, cetyl dimethyl betaine (available as Lonzaine 16SP from Lonza Corp.), lauryl vis-(2-hydroxylethyl)carboxymethyl betaine, oleyl dimethyl gamma-carboxypropyl betaine, lauryl bis-(2-hydroxypropyl)alpha-carboxyethyl betaine, coco dimethyl sulfopropyl betaine, lauryl dimethyl sulfoethyl betaine, lauryl bis-(2-hydroxyethyl)sulfopropyl betaine, amidobetaines, and amidosulfobetaines (wherein the RCONH(CH₂)₃ radical is attached to the nitrogen atom of the betaine), oleyl betain (available as amphoteric Velvetex OLB-50 from Henkel, cocamidopropyl betaine (available as Velvetex BK-53 and BA-35 from Henkel), and mixtures thereof.

Non-limiting examples of sultaines and hydroxysultaines include cocamidopropyl hydroxysultaine (available as Mirataine CBS from Rhone-Poulenc).

Non-limiting examples of other anionic, zwitterionic, amphoteric or optional additional surfactants suitable for use in the compositions are described in McCutcheon's, Emulsifiers and Detergents, 1989 Annual, published by M. C. Publishing Co., and U.S. Pat. Nos. 3,929,678; 2,658,072; 2,438,091; and 2,528,378.

The compositions of the present invention can further comprise additional surfactants for use in combination with the anionic detersive surfactant component described hereinbefore. Suitable optional surfactants include non-ionic and cationic surfactants. Any such surfactant known in the art for use in hair (human or animal) or personal care products can be used, provided that the optional additional surfactant is also chemically and physically compatible with the essential components of the composition, and does not otherwise unduly impair product performance, aesthetics or stability of the composition. The concentration of the optional additional surfactants in the composition can vary with the cleansing or lather performance desired, the optional surfactant selected, the desired product concentration, the presence of other components in the composition, and other factors well known in the art.

Non-limiting examples of suitable non-ionic surfactants include amine oxides, alkyl glucosides, alkyl polyglucosides, polyhydroxy fatty acid amides, alkoxylated fatty acid esters, sugar esters, phosphate esters, ethoxylated esters, glycerol esters, ethoxykates, glycerides, polyethylene glycol 20 sorbitan monolaurate (Polysorbate 20), polyethylene glycol 5 soya sterol, Steareth-20, Ceteareth-20, PPG-2 methyl glucose ether distearate, Ceteth-10, Polysorbate 80, cetyl phosphate, potassium cetyl phosphate, diethanolamine cetyl phosphate, Polysorbate 60, glyceryl stearate, PEG-100 stearate, polyoxyethylene 20 sorbitan trioleate (Polysorbate 85), sorbitan monolaurate, polyoxyethylene 4 lauryl ether sodium stearate, polyglyceryl-4 isostearate, hexyl laurate, diethanolamine cetyl phosphate, glyceryl stearate, and mixtures thereof.

### Cationic Polymer

The compositions of the present invention can contain a cationic polymer to enhance conditioning performance. The concentration of cationic polymers in the compositions of the present invention range from about 0.01% to about 3%, alternatively from about 0.05% to about 2.0%, and alternatively from about 0.1% to about 1.0%, by weight of the composition. Suitable cationic polymers have cationic charge densities of at least about 0.4 meq/gm, alternatively at least about 0.9 meq/gm, alternatively at least about 1.2 meq/gm, but also less than about 7 meq/gm, and alternatively less than about 5 meq/gm, at the pH of intended use of the composition. The pH of intended use of the composition ranges from about pH 3 to about pH 9, and alternatively from about pH 4 to about pH 8.

As used herein, “cationic charge density” of a polymer refers to the ratio of the number of positive charges on the polymer to the molecular weight of the polymer. The average molecular weight of suitable cationic polymers is from about 1,000 to about 10 million, alternatively from about 5,000 to about 5 million, and alternatively from about 10,000 to about 3 million.

Non-limiting examples of suitable cationic polymers for use in the compositions of the present invention contain cationic nitrogen-containing moieties such as quaternary ammonium or cationic protonated amino moieties. The cationic protonated amines can be primary, secondary, or tertiary amines (preferably secondary or tertiary), depending upon the particular species and the selected pH of the composition. Any anionic counterions can be used in association with the cationic polymers as long as the polymers remain soluble in water, in the composition, or in a coacervate phase of the composition, and as long as the counterions are physically and chemically compatible with the essential components of the composition and do not otherwise unduly impair product performance, stability, or aesthetics of the compositions. Non-limiting examples of such counterions include halides (e.g., chloride, fluoride, bromide, iodide), sulfate, methylsulfate, and mixtures thereof. Non-limiting examples of other such cationic polymers are described in the CTFA Cosmetic Ingredient Dictionary, 3rd edition, edited by Estrin, Crosley, and Haynes, (The Cosmetic, Toiletry, and Fragrance Association, Inc., Washington, D.C. (1982).

Non-limiting examples of suitable cationic polymers also include copolymers of vinyl monomers having cationic protonated amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone, vinyl pyrrolidone, and mixtures thereof.

Non-limiting examples of suitable cationic protonated amino and quaternary ammonium monomers, for inclusion in the cationic polymers of the composition herein, include vinyl compounds substituted with dialkylaminoalkyl acrylate, dialkylaminoalkyl methacrylate, monoalkylaminoalkyl acrylate, monoalkylaminoalkyl methacrylate, trialkyl methacryloxyalkyl ammonium salt, trialkyl acryloxyalkyl ammonium salt, diallyl quaternary ammonium salts, and vinyl quaternary ammonium monomers having cyclic cationic nitrogen-containing rings such as pyridinium, imidazolium, and quaternized pyrrolidone, e.g., alkyl vinyl imidazolium, alkyl vinyl pyridinium, alkyl vinyl pyrrolidone salts, and mixtures thereof.

Non-limiting examples of other suitable cationic polymers for use in the compositions of the present invention also include copolymers of 1-vinyl-2-pyrrolidone and 1-vinyl-3-methylimidazolium salt (e.g., chloride salt) (referred to in the industry by the Cosmetic, Toiletry, and Fragrance Association, “CTFA”, as Polyquatemium-16); copolymers of 1-vinyl-2-pyrrolidone and dimethylaminoethyl methacrylate (referred to in the industry by CTFA as Polyquaternium-11); cationic diallyl quaternary ammonium-containing polymers, including, for example, dimethyldiallylammonium chloride homopolymer, copolymers of acrylamide and dimethyldiallylammonium chloride (referred to in the industry by CTFA as Polyquaternium 6 and Polyquaternium 7, respectively); amphoteric copolymers of acrylic acid including copolymers of acrylic acid and dimethyldiallylammonium chloride (referred to in the industry by CTFA as Polyquaternium 22), terpolymers of acrylic acid with dimethyldiallylammonium chloride and acrylamide (referred to in the industry by CTFA as Polyquaternium 39), and terpolymers of acrylic acid with methacrylamidopropyl trimethylammonium chloride, methylacrylate (referred to in the industry by CTFA as Polyquaternium 47), and mixtures thereof.

Non-limiting examples of particularly suitable cationic substituted monomers include the cationic substituted dialkylaminoalkyl acrylamides, dialkylaminoalkyl methacrylamides, and mixtures thereof. These monomers conform to the Formula (II):

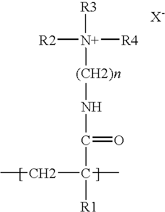

wherein R¹ is hydrogen, methyl or ethyl; each of R², R³ and R⁴ are independently hydrogen or a short chain alkyl having from about 1 to about 8 carbon atoms, alternatively from about 1 to about 5 carbon atoms, and alternatively from about 1 to about 2 carbon atoms; n is an integer having a value of from about 1 to about 8, and alternatively from about 1 to about 4; and X is a counterion. The nitrogen attached to R², R³ and R⁴ may be a protonated amine (primary, secondary or tertiary), but is preferably a quaternary ammonium wherein each of R², R³ and R⁴ are alkyl groups, a non-limiting example of which is polymethyacrylamidopropyl trimonium chloride, available under the trade name Polycare 133, from Rhone-Poulenc, Cranberry, N.J., U.S.A.

Non-limiting examples of still other suitable cationic polymers for use in the compositions of the present invention include polysaccharide polymers, such as cationic cellulose derivatives and cationic starch derivatives and mixtures thereof. Non-limiting examples of suitable cationic polysaccharide polymers include those which conform to the Formula (III):

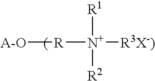

wherein A is an anhydroglucose residual group, such as a starch or cellulose anhydroglucose residual; R is an alkylene oxyalkylene, polyoxyalkylene, or hydroxyalkylene group, or mixtures thereof; R1, R2, and R3 independently are alkyl, aryl, alkylaryl, arylalkyl, alkoxyalkyl, or alkoxyaryl groups, each group containing up to about 18 carbon atoms, and the total number of carbon atoms for each cationic moiety (i.e., the sum of carbon atoms in R1, R2 and R3) being about 20 or less; and X is an anionic counterion as described hereinbefore.

Non-limiting examples of cationic cellulose polymers particularly useful in compositions of the present invention include salts of hydroxyethyl cellulose reacted with trimethyl ammonium substituted epoxide, referred to in the industry (CTFA) as Polyquaternium 10 and available from Amerchol Corp. (Edison, N.J., USA) in their Polymer LR, JR, and KG series of polymers.

Non-limiting examples of other suitable types of cationic cellulose include the polymeric quaternary ammonium salts of hydroxyethyl cellulose reacted with lauryl dimethyl ammonium-substituted epoxide referred to in the industry (CTFA) as Polyquatemium 24. These materials are available from Amerchol Corp. under the tradename Polymer LM-200.

Non-limiting examples of other suitable cationic polymers include cationic guar gum derivatives, such as guar hydroxypropyltrimonium chloride, examples of which include the Jaguar series commercially available from Rhone-Poulenc Incorporated and the N-Hance series commercially available from Aqualon Division of Hercules, Inc.

Non-limiting examples of other suitable cationic polymers include quaternary nitrogen-containing cellulose ethers, examples of which are described in U.S. Pat. No. 3,962,418.

Non-limiting examples of still other suitable cationic polymers include copolymers of etherified cellulose, guar and starch, examples of which are described in U.S. Pat. No. 3,958,581.

When used, the cationic polymers herein are either soluble in the composition or are soluble in a complex coacervate phase in the composition formed by the cationic polymer and the anionic, amphoteric and/or zwitterionic detersive surfactant component described hereinbefore. Complex coacervates of the cationic polymer can also be formed with other charged materials in the composition.

Techniques for analysis of formation of complex coacervates are known in the art. For example, microscopic analyses of the compositions, at any chosen stage of dilution, can be utilized to identify whether a coacervate phase has formed. Such coacervate phase will be identifiable as an additional emulsified phase in the composition. The use of dyes can aid in distinguishing the coacervate phase from other insoluble phases dispersed in the composition.

### II. Conditioning Agents

The compositions of the present invention also include conditioning agents. The performance of the conditioning agents can be enhanced by a cationic polymer as hereinbefore described. As used herein, the term “conditioning agent” includes any material which is used to give a particular conditioning benefit to hair and/or skin. In human and/or animal hair treatment compositions, suitable conditioning agents are those which deliver one or more benefits relating to shine, softness, combability, antistatic properties, wet-handling, damage, manageability, body, and greasiness. Conditioning agents useful in the compositions of the present invention comprise a water-insoluble, water dispersible, non-volatile liquid that forms emulsified liquid particles.

The conditioning agents are present in the compositions of the present inventions in the range of from about 0.01% to about 10% and alternatively from about 0.05% to about 8%, by weight of the composition. However, the concentration of the conditioning agent in the composition should be sufficient to provide the desired conditioning benefits, as will be apparent to one of ordinary skill in the art. Such concentration can vary with the conditioning agent, the conditioning performance desired, the average size of the conditioning agent particles, the type and concentration of other components, and other like factors.

Non-limiting examples of suitable conditioning agents for use in the composition include conditioning agents characterized generally as silicones (e.g., silicone oils, cationic silicones, silicone gums, high refractive silicones, silicone resins, silicone polymers, and mixtures thereof), organic conditioning oils (e.g., hydrocarbon oils, polyolefins, fatty esters, and mixtures thereof) and mixtures thereof, or those conditioning agents which otherwise form liquid, dispersed particles in the aqueous surfactant matrix herein. Such conditioning agents should be physically and chemically compatible with the essential components of the composition, and should not otherwise unduly impair product stability, aesthetics or performance.

Additional non-limiting examples of suitable conditioning agents include fatty acids, esters of fatty acids, fatty alcohols, ethoxylated alcohols, polyol polyesters, glycerin, glycerin monoesters, glycerin polyesters, epidermal and sebaceous hydrocarbons, lanolin, straight and branched chain hydrocarbons, vegetable oils, vegetable oil adduct, hydrogenated vegetable oils, non-ionic polymers, natural waxes, synthetic waxes, petrolatum, petrolatum derivatives, polyolefinic glycols, polyolefinic mono-esters, polyolefinic polyesters, cholesterols, cholesterol esters, paraffin, mineral oil, stearyl alcohol, cetyl alcohol, cetearyl alcohol, behenyl alcohol, C₁₀-C₃₀ polyesters of sucrose, stearic acid, palmitic acid, behenic acid, oleic acid, linoleic acid, myristic acid, lauric acid, ricinoleic acid, steareth-1-100, cetereath-1-100, clyceryl tribehenate, glyceryl dipalmitate, glyceryl monostearate, trihydroxystearin, zozkerite wax, jojoba wax, lanolin wax, ethylene glycol distearate, candelilla wax, carnauba wax, beeswax, disilicone waxes, and mixtures thereof.

a. Silicones

The conditioning agent of the compositions of the present invention is preferably an insoluble silicone conditioning agent. The silicone conditioning agent particles can comprise volatile silicone, non-volatile silicone, and mixtures thereof. Conditioning agents particularly useful in the compositions of the present invention are non-volatile silicone conditioning agents. If volatile silicones are present, such presence will typically be incidental and due to their use as a solvent or carrier for commercially available forms of non-volatile silicone materials ingredients, such as silicone gums and resins. The silicone conditioning agent particles can comprise a silicone fluid conditioning agent and can also comprise other ingredients, such as a silicone resin to improve silicone fluid deposition efficiency and/or enhance glossiness of the hair.

The silicone conditioning agent is present in the compositions of the present invention at a concentration of from about 0.01% to about 10%, alternatively from about 0.1% to about 8%, alternatively from about 0.1% to about 5%, and alternatively from about 0.2% to about 3%, by weight of the composition. Non-limiting examples of suitable silicone conditioning agents, and optional suspending agents for the silicone, are described in U.S. Reissue Pat. No. 34,584, U.S. Pat. No. 5,104,646, and U.S. Pat. No. 5,106,609. The silicone conditioning agents for use in the compositions of the present invention have a viscosity, as measured at 25° C., of from about 20 to about 2,000,000 centistokes (“csk”), alternatively from about 1,000 to about 1,800,000 csk, alternatively from about 50,000 to about 1,500,000 csk, and alternatively from about 100,000 to about 1,500,000 csk.

The dispersed silicone conditioning agent particles of the compositions of the present invention have a number average particle diameter of from about 0.01 μm to about 50 μm. For small particle application to hair, the number average particle diameters range from about 0.01 μm to about 10 μm, alternatively from about 0.01 μm to about 5 μm, and alternatively from about 0.01 μm to about 3 μm. For larger particle application to hair, the volume average particle diameters range from about 5 μm to about 125 μm, alternatively from about 10 μm to about 90 μm, alternatively from about 15 μm to about 70 μm, and alternatively from about 20 μm to about 50 μm. Conditioning agents having an average particle size of less than about 5 μm may deposit more efficiently on the hair. It is believed that small size particles of conditioning agent are contained within the coacervate that is formed between the anionic surfactant component and the cationic polymer component upon dilution of the shampoo. Larger particles of conditioning agents may deposit independently, though less efficiently.

General description of silicones including sections description of silicone fluids, gums, and resins, as well as manufacture of silicones, is found in Encyclopedia of Polymer Science and Engineering, vol. 15, 2d ed., pp 204-308, John Wiley & Sons, Inc. (1989).

i. Silicone Oils

Suitable silicone fluids include silicone oils, which are flowable silicone materials having a viscosity, as measured at 25° C., of less than 1,000,000 csk, alternatively from about 5 csk to about 1,000,000 csk, and alternatively from about 100 csk to about 600,000 csk. Non-limiting examples of silicone oils suitable for use in the compositions of the present invention include polyalkyl siloxanes, polyaryl siloxanes, polyalkylaryl siloxanes, polyether siloxane copolymers, and mixtures thereof. Other insoluble, non-volatile silicone fluids having hair conditioning properties can also be used.

Silicone oils include polyalkyl or polyaryl siloxanes which conform to the following Formula (IV):

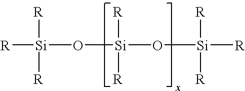

wherein R is aliphatic, preferably alkyl or alkenyl, or aryl, R can be substituted or unsubstituted, and x is an integer from 1 to about 8,000. Non-limiting examples of R groups suitable for use in the compositions of the present invention include: alkoxy, aryloxy, alkaryl, arylalkyl, arylalkenyl, alkamino, and ether-substituted, hydroxyl-substituted, and halogen-substituted aliphatic and aryl groups. Suitable R groups also include cationic amines and quaternary ammonium groups.

Particularly useful alkyl and alkenyl substituents include C₁ to C₅ alkyls and alkenyls, alternatively C₁ to C₄, and alternatively C₁ to C₂. The aliphatic portions of other alkyl-, alkenyl-, or alkynyl-containing groups (such as alkoxy, alkaryl, and alkamino) can be straight or branched chains, and are from C₁ to C₅, alternatively C₁ to C₄, alternatively C₁ to C₃, and alternatively C₁ to C₂. As discussed above, the R substituents can also contain amino functionalities (e.g. alkamino groups), which can be primary, secondary or tertiary amines or quaternary ammonium. These include mono-, di- and tri-alkylamino and alkoxyamino groups, wherein the aliphatic portion chain length is as described herein.

ii. Amino and Cationic Silicones

Non-limiting examples of cationic silicone fluids suitable for use in the compositions of the present invention include those which conform to the general Formula (V):

(R₁)ₐG₃₋ₐ-Si—(—OSiG₂)ₙ-(—OSiG_{b}(R₁)_{2-b)m}—O—SiG₃₋ₐ(R₁)ₐ

wherein G is hydrogen, phenyl, hydroxy, or C₁-C₈ alkyl, preferably methyl; a is 0 or an integer having a value from 1 to 3, preferably 0; b is 0 or 1, preferably 1; n is a number from 0 to 1,999, alternatively from 49 to 499; m is an integer from 1 to 2,000, alternatively from 1 to 10; the sum of n and m is a number from 1 to 2,000, alternatively from 50 to 500; R₁ is a monovalent radical conforming to the general formula CqH_{2q}L, wherein q is an integer having a value from 2 to 8, and L is selected from the following groups:

—N(R₂)CH₂—CH₂—N(R₂)₂

—N(R₂)₂

—N(R₂)₃A⁻

—N(R₂)CH₂—CH₂—NR₂H₂A⁻

wherein R₂ is hydrogen, phenyl, benzyl, or a saturated hydrocarbon radical, preferably an alkyl radical from about C₁ to about C₂₀, and A⁻ is a halide ion.

A particular example of a cationic silicone corresponding to Formula (V) is the polymer known as “trimethylsilylamodimethicone”, which is shown below in Formula (VI):

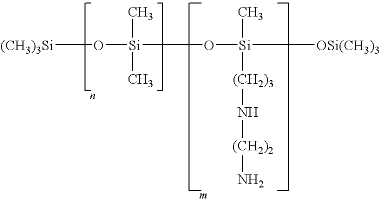

Other silicone cationic polymers which may be used in the compositions of the present invention are represented by the general Formula (VII):

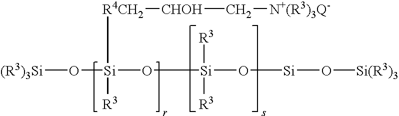

wherein R³ is a monovalent hydrocarbon radical from C₁ to C₁₈, particularly an alkyl or alkenyl radical, such as methyl; R₄ is a hydrocarbon radical, particularly a C₁ to C₁₈ alkylene radical or a C₁₀ to C₁₈ alkyleneoxy radical, and alternatively a C₁ to C₈ alkyleneoxy radical; Q⁻ is a halide ion, particularly chloride; r is an average statistical value from 2 to 20, and alternatively from 2 to 8; s is an average statistical value from 20 to 200, and alternatively from 20 to 50.
iii. Silicone Gums

Non-limiting examples of other silicone fluids suitable for use in the compositions of the present invention include insoluble silicone gums. These gums are polyorganosiloxane materials having a viscosity, as measured at 25° C., of greater than or equal to 1,000,000 csk. Silicone gums are described in U.S. Pat. No. 4,152,416; Noll and Walter, Chemistry and Technology of Silicones, New York: Academic Press (1968); and in General Electric Silicone Rubber Product Data Sheets SE 30, SE 33, SE 54 and SE 76. Non-limiting examples of silicone gums for use in the compositions of the present invention include polydimethylsiloxane, (polydimethylsiloxane) (methylvinylsiloxane) copolymer, poly(dimethylsiloxane) (diphenyl siloxane)(methylvinylsiloxane) copolymer and mixtures thereof.

iv. High Refractive Index Silicones

Non-limiting examples of non-volatile, insoluble silicone fluid conditioning agents suitable for use in the compositions of the present invention include those known as “high refractive index silicones,” having a refractive index of at least about 1.46, alternatively at least about 1.48, alternatively at least about 1.52, and alternatively at least about 1.55. The refractive index of the polysiloxane fluid is generally less than about 1.70, and alternatively less than about 1.60. As used herein, polysiloxane “fluid” includes oils as well as gums.

The high refractive index polysiloxane fluid includes those represented by general Formula (IV) above, as well as cyclic polysiloxanes such as those represented by Formula (VIII) below:

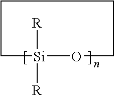

wherein R is as defined above, and n is a number from about 3 to about 7, and alternatively from about 3 to about 5.

The high refractive index polysiloxane fluids contain an amount of aryl-containing R substituents sufficient to increase the refractive index to the desired level, which is described herein. Additionally, R and n are selected so that the material is non-volatile.

Aryl-containing substituents include those which contain alicyclic and heterocyclic five and six member aryl rings and those which contain fused five or six member rings. The aryl rings themselves can be substituted or unsubstituted.

Generally, the high refractive index polysiloxane fluids suitable for use in the compositions of the present invention have a degree of aryl-containing substituents of at least about 15%, alternatively at least about 20%, alternatively at least about 25%, alternatively at least about 35%, and alternatively at least about 50%. The degree of aryl substitution is less than about 90%, alternatively less than about 85%, and alternatively from about 55% to about 80%.

Preferred high refractive index polysiloxane fluids have a combination of phenyl or phenyl derivative substituents (more preferably phenyl), with alkyl substituents, preferably C₁-C₄ alkyl (more preferably methyl), hydroxy, or C₁-C₄ alkylamino (especially —R¹NHR²NH2 wherein each R¹ and R² independently is a C₁-C₃ alkyl, alkenyl, and/or alkoxy).

When high refractive index silicones are used in the compositions of the present invention, they are used in solution with a spreading agent, such as a silicone resin or a surfactant, to reduce the surface tension by an amount sufficient to enhance spreading and thereby enhance the glossiness (subsequent to drying) of hair treated with the compositions.

Silicone fluids suitable for use in the compositions of the present invention are described in U.S. Pat. No. 2,826,551, U.S. Pat. No. 3,964,500, U.S. Pat. No. 4,364,837, British Pat. No. 849,433, and Silicon Compounds, Petrarch Systems, Inc. (1984).

v. Silicone Resins

Silicone resins can be included in the silicone conditioning agent of the compositions of the present invention. Suitable resins include highly cross-linked polymeric siloxane systems. The cross-linking is introduced through the incorporation of trifunctional and tetrafunctional silanes with monofunctional or difunctional, or both, silanes during manufacture of the silicone resin.

Silicone materials and silicone resins in particular, can conveniently be identified according to a shorthand nomenclature system known to those of ordinary skill in the art as “MDTQ” nomenclature. Under this system, the silicone is described according to presence of various siloxane monomer units which make up the silicone. Briefly, the symbol M denotes the monofunctional unit (CH₃)₃SiO_{0.5}; D denotes the difunctional unit (CH₃)₂SiO; T denotes the trifunctional unit (CH₃)SiO_{1.5}; and Q denotes the quadra- or tetra-functional unit SiO₂. Primes of the unit symbols (e.g. M′, D′, T′, and Q′) denote substituents other than methyl, and must be specifically defined for each occurrence.

Non-limiting examples of silicone resins particularly suitable for use in the compositions of the present invention include MQ, MT, MTQ, MDT and MDTQ resins. Methyl is a preferred silicone substituent. Particularly useful silicone resins are MQ resins, wherein the M:Q ratio is from about 0.5:1.0 to about 1.5:1.0, and the average molecular weight of the silicone resin is from about 1000 to about 10,000.

b. Organic Conditioning Oils

The conditioning component of the compositions of the present invention can also comprise from about 0.05% to about 3%, alternatively from about 0.08% to about 1.5%, and alternatively from about 0.1% to about 1%, by weight of the composition, of at least one organic conditioning oil as the conditioning agent, either alone or in combination with other conditioning agents, such as the silicones (described hereinabove).

i. Hydrocarbon Oils

Non-limiting examples of suitable organic conditioning oils for use as conditioning agents in the compositions of the present invention include hydrocarbon oils having at least about 10 carbon atoms, such as cyclic hydrocarbons, straight chain aliphatic hydrocarbons (saturated or unsaturated), and branched chain aliphatic hydrocarbons (saturated or unsaturated), including polymers, and mixtures thereof. Suitable straight chain hydrocarbon oils are from about C₁₂ to about C₁₉. Suitable branched chain hydrocarbon oils, including hydrocarbon polymers, can contain more than about 19 carbon atoms.

Non-limiting examples of suitable hydrocarbon oils include paraffin oil, mineral oil, saturated and unsaturated dodecane, saturated and unsaturated tridecane, saturated and unsaturated tetradecane, saturated and unsaturated pentadecane, saturated and unsaturated hexadecane, polybutene, polydecene, and mixtures thereof. Branched-chain isomers of these compounds, as well as of higher chain length hydrocarbons, can also be used, examples of which include highly branched, saturated or unsaturated, alkanes such as the permethyl-substituted isomers, e.g., the permethyl-substituted isomers of hexadecane and eicosane, such as 2,2,4,4,6,6,8,8-dimethyl-10-methylundecane and 2,2,4,4,6,6-dimethyl-8-methylnonane, available from Permethyl Corporation, and mixtures thereof. Hydrocarbon polymers such as polybutene and polydecene can also be used. A particularly suitable hydrocarbon polymer is polybutene, such as the copolymer of isobutylene and butene. A commercially available material of this type is L-14 polybutene from Amoco Chemical Corporation. The concentration of such hydrocarbon oils in the composition ranges from about 0.05% to about 20%, alternatively from about 0.08% to about 1.5%, and alternatively from about 0.1% to about 1%, by weight of the composition.

ii. Polyolefins

Organic conditioning oils for use in the compositions of the present invention can also include liquid polyolefins, liquid poly-α-olefins, and hydrogenated liquid poly-α-olefins. Polyolefins for use herein are prepared by polymerization of C₄ to about C₁₄ olefinic monomers, and alternatively from olefinic monomers from about C₆ to about C₁₂.

Non-limiting examples of olefinic monomers suitable for use in preparing the polyolefin liquids described herein include ethylene, propylene, 1-butene, 1-pentene, 1-hexene, 1-octene, 1-decene, 1-dodecene, 1-tetradecene, branched chain isomers such as 4-methyl-1-pentene, and mixtures thereof. Also suitable for preparing the polyolefin liquids are olefin-containing refinery feedstocks or effluents. Non-limiting examples of particularly suitable hydrogenated α-olefin monomers include 1-hexene to 1-hexadecenes, 1-octene to 1-tetradecene, and mixtures thereof.

iii. Fatty Esters

Non-limiting examples of other suitable organic conditioning oils for use as the conditioning agent in the compositions of the present invention include fatty esters having at least 10 carbon atoms. These fatty esters include esters with hydrocarbyl chains derived from fatty acids or alcohols (e.g. mono-esters, polyhydric alcohol esters, and di- and tri-carboxylic acid esters). The hydrocarbyl radicals of the fatty esters hereof can include, or have covalently bonded thereto, other compatible functionalities, such as amides and alkoxy moieties (e.g., ethoxy or ether linkages, etc.).

Non-limiting examples of particularly suitable fatty esters include isopropyl isostearate, hexyl laurate, isohexyl laurate, isohexyl palmitate, isopropyl palmitate, decyl oleate, isodecyl oleate, hexadecyl stearate, decyl stearate, isopropyl isostearate, dihexyldecyl adipate, lauryl lactate, myristyl lactate, cetyl lactate, oleyl stearate, oleyl oleate, oleyl myristate, lauryl acetate, cetyl propionate, and oleyl adipate, and mixtures thereof.

Non-limiting examples of other fatty esters suitable for use in the compositions of the present invention include mono-carboxylic acid esters of the general formula R′COOR, wherein R′ and R are alkyl or alkenyl radicals, and the sum of carbon atoms in R′ and R is at least 10, and alternatively at least 22.

Non-limiting examples of still other fatty esters suitable for use in the compositions of the present invention include di- and tri-alkyl and alkenyl esters of carboxylic acids, such as esters of C₄ to C₈ dicarboxylic acids (e.g. C₁ to C₂₂ esters, alternatively C₁ to C₆, of succinic acid, glutaric acid, and adipic acid). Non-limiting examples of di- and tri-alkyl and alkenyl esters of carboxylic acids include isocetyl stearyol stearate, diisopropyl adipate, tristearyl citrate, and mixtures thereof.

Other fatty esters suitable for use in the compositions of the present invention are those known as polyhydric alcohol esters. Non-limiting examples of such polyhydric alcohol esters include alkylene glycol esters, such as ethylene glycol mono and di-fatty acid esters, diethylene glycol mono- and di-fatty acid esters, polyethylene glycol mono- and di-fatty acid esters, propylene glycol mono- and di-fatty acid esters, polypropylene glycol monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, polyoxyethylene sorbitan fatty acid esters, and mixtures thereof.

Non-limiting examples of still other fatty esters suitable for use in the compositions of the present invention include glycerides, including, mono-, di-, and tri-glycerides, alternatively di- and tri-glycerides, and alternatively triglycerides. Glycerides particularly suitable for use in the compositions described herein are the mono-, di-, and tri-esters of glycerol and long chain carboxylic acids, such as C₁₀ to C₂₂ carboxylic acids. A variety of these types of materials can be obtained from vegetable and animal fats and oils, such as castor oil, safflower oil, cottonseed oil, corn oil, olive oil, cod liver oil, almond oil, avocado oil, palm oil, sesame oil, lanolin and soybean oil, and mixtures thereof. Non-limiting examples of suitable synthetic oils include triolein and tristearin glyceryl dilaurate, and mixtures thereof.

Other fatty esters suitable for use in the compositions of the present invention are water insoluble synthetic fatty esters. Particularly suitable synthetic esters conform to the general Formula (IX):

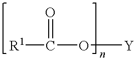

wherein R¹ is a C₇ to C₉ alkyl, alkenyl, hydroxyalkyl or hydroxyalkenyl group, alternatively a saturated alkyl group, and alternatively a saturated, linear, alkyl group; n is a positive integer having a value from 2 to 4, and alternatively 3; and Y is an alkyl, alkenyl, hydroxy or carboxy substituted alkyl or alkenyl, having from about 2 to about 20 carbon atoms, and alternatively from about 3 to about 14 carbon atoms.

Other suitable synthetic esters conform to the general Formula (X):

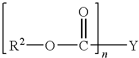

wherein R² is a C₈ to C₁₀ alkyl, alkenyl, hydroxyalkyl or hydroxyalkenyl group; alternatively a saturated alkyl group, alternatively a saturated, linear, alkyl group; n and Y are as defined above in Formula (X).

Non-limiting examples of suitable synthetic fatty esters for use in the compositions of the present invention include P-43 (C₈-C₁₀ triester of trimethylolpropane), MCP-684 (tetraester of 3,3 diethanol-1,5 pentadiol), MCP 121 (C₈-C₁₀ diester of adipic acid), and mixtures thereof, all of which are available from Mobil Chemical Company.

c. Other Conditioning Agents

Also suitable for use in the compositions herein are the conditioning agents described in U.S. Pat. Nos. 5,674,478, and 5,750,122. Also suitable for use herein are those conditioning agents described in U.S. Pat. Nos. 4,529,586; 4,507,280; 4,663,158; 4,197,865; 4,217,914; 4,381,919; and 4,422,853.

### III. Additional Components

The compositions of the present invention can also optionally comprise a range of additional components. Such components should be pharmaceutically acceptable and compatible with the necessary components of the compositions of the present invention. The CTFA Cosmetic Ingredient Handbook, Second Edition, 1992, describes non-limiting examples of such components. Non-limiting examples of classes of such components include abrasives, absorbents, antioxidants, binders, biological additives, buffering agents, bulking agents, chemical additives, colorants, fragrances, pigments, cosmetic biocides, denaturants, drug astringents, external analgesics, film formers, fragrance components, humectants, opacifying agents, pH adjusting agents, chelating agents, mono- or divalent satls (such as sodium chloride) preservatives, propellants, reducing agents, essential oils, skin sensates, skin soothing agents, skin healing agents, skin moisturizing agents, and combinations thereof.

### Treatment Agent

The present invention can also include additional components such as various treatment agents. Non-limiting examples of such treatment agents include vitamins, peptides, desquamation actives, anti-atrophy actives, anti-oxidants, flavanoids, anti-inflammatory agents, topical anesthetics, chelators, antimicrobial actives, anti-fungal actives, soothing actives, healing actives, moisturizing actives, flea actives, tick actives, other insect actives, fragrances, and mixtures thereof.

### Suspending Agent

The compositions of the present invention can further comprise a suspending agent at concentrations effective for suspending the polymeric liquid crystal or the dispersed particles of a water insoluble, conditioning agent, or other water-insoluble, dispersed material in the compositions. Such concentrations range from about 0.1% to about 10%, and alternatively from about 0.3% to about 5.0%, by weight of the compositions.

Suitable suspending agents include crystalline suspending agents that can be categorized as acyl derivatives, long chain amine oxides, or combinations thereof. These suspending agents are described in U.S. Pat. No. 4,741,855. Non-limiting examples of suitable suspending agents include ethylene glycol esters of fatty acids having from about 16 to about 22 carbon atoms. Non-limiting examples of particularly suitable suspending agents are ethylene glycol stearates, both mono and distearate, but particularly the distearate containing less than about 7% of the mono stearate. Non-limiting examples of other suitable suspending agents include alkanol amides of fatty acids, having from about 16 to about 22 carbon atoms, and alternatively from about 16 to about 18 carbon atoms. Non-limiting examples of particularly suitable suspending agents include stearic monoethanolamide, stearic diethanolamide, stearic monoisopropanolamide, stearic monoethanolamide stearate, and mixtures thereof.

Non-limiting examples of other suitable suspending agents include long chain acyl derivatives such as long chain esters of long chain fatty acids (e.g., stearyl stearate, cetyl palmitate, etc.); glyceryl esters (e.g., glyceryl distearate), long chain esters of long chain alkanol amides (e.g., stearamide diethanolamide distearate, stearamide monoethanolamide stearate), and mixtures thereof. Non-limiting examples of other suitable suspending agents include long chain acyl derivatives, ethylene glycol esters of long chain carboxylic acids, long chain amine oxides, and alkanol amides of long chain carboxylic acids, and mixtures thereof. For example, it is contemplated that suspending agents with long chain hydrocarbyls having C₈-C₂₂ chains can be used.

Non-limiting examples of other long chain acyl derivatives suitable for use as suspending agents include N,N-dihydrocarbyl amido benzoic acid and soluble salts thereof (e.g., Na, K), N,N-di(hydrogenated) C₁₆, C₁₈, and tallow amido benzoic acid species of this family, which are commercially available from Stepan Company (Northfield, Ill., USA).

Non-limiting examples of suitable long chain amine oxides for use as suspending agents include alkyl (C₁₆-C₂₂) dimethyl amine oxides, e.g., stearyl dimethyl amine oxide.

Other suitable suspending agents include primary amines having a fatty alkyl moiety having at least about 16 carbon atoms, non-limiting examples of which include palmitamine, stearamine and mixtures thereof, and secondary amines having two fatty alkyl moieties each having at least about 12 carbon atoms, non-limiting examples of which include dipalmitoylamine, di(hydrogenated tallow) amine, and mixtures thereof.

Non-limiting examples of still other suitable suspending agents include di(hydrogenated tallow)phthalic acid amide, crosslinked maleic anhydride-methyl vinyl ether copolymer, and mixtures thereof.

### Friction Test

It has been found that hair conditioning level corresponds with the friction coefficient of hair wherein highly conditioned hair demonstrates a low friction coefficient while less conditioned hair demonstrates a higher friction coefficient. Friction coefficient can therefore be indicative of the level of conditioning a composition provides. The friction coefficient as measured herein, and the corresponding conditioning levels, can be correlated to the desired conditioning levels and coat characteristics previously described.

After treatment of hair with the compositions of the present invention, the compositions provide a friction coefficient of from about 0.65 to about 0.8, alternatively from about 0.68 to about 0.77, alternatively from about 0.7 to about 0.75

The friction coefficient is determined according to the following method:

The method measures the change in friction of an untreated versus treated hair switch. A weighted “sled” (approximately 4.6 cm×3.1 cm) weighing 73 g+/−5% and covered with a nylon mesh (100 micron nylon mesh available from Small Parts, Inc. Dist. Div., Miramar, Fla. USA) of approximately 105 micron pore size is attached, using a polyethylene line (Spiderwire, 501b, 101b Test Dia., Spirit Lake, Iowa USA), to a force measurement device such as an Instron model 5542 and pulled at a constant speed along the length of a 20 g hair switch (approximately 10 inches or 25.4 cm in length) that is clamped on one end. The hair switch is about 3 inches (7.6 cm) wide.

The hair that is used in the test method is brown Caucasian hair that has been formed into 20 gram switches that are ten inches (25.4 cm) in length, and which has been cleaned to remove any foreign soils. The hair switches are then allowed to equilibrate in a constant temperature room at 75° C./50% relative humidity (RH) overnight.

The hair switches are treated as follows if treated with composition to be tested:

The switches are pre-wetted under running tap water. The excess water is squeezed out and the switch is hung on a rack. For leave-on products, 1.5 cc (1.5 mL) of the test product is then applied to the surface of the hair switch and rubbed into the switch for about 30-40 seconds. For rinse-off products, the test product is applied to the wet hair at a dose of 0.1 g/g and massaged/lathered for approximately 30 seconds followed by approximately 30 seconds of water rinsing at a flow rate of approximately 1.5 gallon/minute, and the process is repeated to complete one treatment cycle.

The switches are then dried (by air drying, in a dry box, or blown dry, with one drying method used consistently throughout the method) and the previously described treatment cycle is repeated for a total of 3 cycles. After treatment, the switches are re-hung on the rack and placed in a constant temperature room (75° C./50% RH) to equilibrate overnight.

Treated and untreated switches are tested as follows:

The switch to be tested is clamped into position on a level horizontal testing stand and combed 2-3 times to orient the hair and remove tangles. The “sled” is then attached to the Instron 5542 with a polyethylene line (Spiderwire, 501b, 101b Test Dia.) and placed on the hair switch. Friction is measured on three separate switches in the forward direction (toward tip end of hair) by pulling the “sled” at a rate of approximately 1 cm/sec while measuring the tension force (typically measured in grams). Each force measurement is an average of at least ten values recorded over a distance of at least 5 cm once the “sled” has reached constant speed. A minimum of three measurements is taken for each switch.

The friction coefficient is determined as the average ratio of the friction of the treated hair switch divided by the friction of an untreated hair switch.

### Methods of Making

The compositions of the present invention may be made by any appropriate method known to those of skill in the art for making cleansing and/or conditioning compositions. In general, the compositions of the present invention may be made by mixing the ingredients together at either room temperature or at elevated temperature, e.g. about 72° C. Heat is only required if solid ingredients are in the composition. The ingredients are mixed together at the batch processing temperature (either room temperature or elevated temperature). Additional ingredients may be added to the product at room temperature.

### EXAMPLES

Short Coat Examples

| EXAMPLE COMPOSITION | 1 | 2 | 3 | 4 | 5 |
|---|---|---|---|---|---|
| Ammonium Laureth Sulfate | 8.00 | 7.00 | 9.00 | 10.00 | |
| (AE₃S) | | | | | |
| Ammonium Lauryl Sulfate | 8.00 | 9.00 | 7.00 | 6.00 | |
| (ALS) | | | | | |
| Sodium Laureth Sulfate | | | | | 9.00 |
| (SLE₃S) | | | | | |
| Sodium Lauryl Sulfate | | | | | 7.00 |
| (SLS) | | | | | |
| Cocamide MEA | 0.80 | 0.80 | 0.80 | 0.80 | 0.80 |
| Cetyl Alcohol | 0.90 | 0.90 | 0.90 | 0.90 | 0.90 |
| Polyquaternium-10 (1) | 0.20 | | 0.20 | 0.10 | |
| Polyquaternium-10 (2) | | | | | 0.20 |
| Guar Hydroxypropyltrimonium | | 0.13 | | | |
| Chloride (3) | | | | | |
| Dimethicone (4) | 1.35 | 1.50 | 1.35 | 1.00 | 1.25 |
| PEG 7M | 0.10 | 0.10 | 0.10 | 0.10 | 0.10 |
| Ethylene Glycol Distearate | 1.50 | 1.50 | 1.50 | 1.50 | 1.50 |
| Fragrance | 0.70 | 0.70 | 0.70 | 0.70 | 0.70 |
| Sodium Chloride | 0.50 | 0.50 | 0.50 | 0.50 | 0.50 |
| Citric Acid | 0.04 | 0.04 | 0.04 | 0.04 | 0.04 |
| Sodium Citrate | 0.4 | 0.4 | 0.4 | 0.4 | 0.4 |
| Sodium Benzoate | 0.25 | 0.25 | 0.25 | 0.25 | 0.25 |
| Ethylene Diamine Tetra | 0.10 | 0.10 | 0.10 | 0.10 | 0.10 |
| Acetic Acid | | | | | |
| Water and Minors | QS to 100% | QS to 100% | QS to 100% | QS to 100% | QS to 100% |
| (QS to 100%) | | | | | |
| Friction Coefficient | 0.77 | 0.75 | 0.78 | 0.80 | 0.79 |
| Provided | | | | | |

(1) Polymer LR400M available from Amerchol [charge density = 0.7 meq/g, molecular weight ~400,000]
(2) Polymer LR30M available from Amerchol [charge density = 0.7 meq/g, molecular weight ~1,800,000]
(3) N-Hance 3270 available from Hercules (Aqualon), [charge density = 0.7 meq/g, molecular weight ~500,000]
(4) Viscasil 330M (General Electric Silicones)

Long Coat Examples

| EXAMPLE COMPOSITION | 6 | 7 | 8 | 9 | 10 | 11 |
|---|---|---|---|---|---|---|
| Ammonium Laureth Sulfate | 7.00 | 10.00 | 9.00 | 8.00 | 6.00 | |
| (AE₃S) | | | | | | |
| Ammonium Lauryl Sulfate | 9.00 | 6.00 | 7.00 | 8.00 | 10.00 | |
| (ALS) | | | | | | |
| Sodium Laureth Sulfate | | | | | | 8.00 |
| (SLE₃S) | | | | | | |
| Sodium Lauryl Sulfate | | | | | | 8.00 |
| (SLS) | | | | | | |
| Cocamide MEA | 0.80 | 0.80 | 0.80 | 0.80 | 0.80 | 0.80 |
| Cetyl Alcohol | 0.90 | 0.90 | 0.90 | 0.90 | 0.90 | 0.90 |
| Polyquaternium-10 (1) | 0.10 | | | | | |
| Polyquaternium-10 (2) | | 0.50 | | | | |
| Guar Hydroxypropyltrimonium | | | | 0.10 | | |
| Chloride (3) | | | | | | |
| Dimethicone (4) | 3.00 | 4.35 | 3.00 | 2.50 | 3.00 | 2.00 |
| PEG 7M | 0.10 | 0.10 | 0.10 | 0.10 | 0.10 | 0.10 |
| Ethylene Glycol Distearate | 1.50 | 1.50 | 1.50 | 1.50 | 1.50 | 1.50 |
| Fragrance | 0.70 | 0.70 | 0.70 | 0.70 | 0.70 | 0.70 |
| Sodium Chloride | 0.50 | 0.50 | 0.50 | 0.50 | 0.50 | 0.50 |
| Citric Acid | 0.04 | 0.04 | 0.04 | 0.04 | 0.04 | 0.04 |
| Sodium Citrate | 0.40 | 0.4 | 0.4 | 0.4 | 0.4 | 0.4 |
| Sodium Benzoate | 0.25 | 0.25 | 0.25 | 0.25 | 0.25 | 0.25 |
| Ethylene Diamine Tetra | 0.10 | 0.10 | 0.10 | 0.10 | 0.10 | 0.10 |
| Acetic Acid | | | | | | |
| Water and Minors | QS to 100% | QS to 100% | QS to 100% | QS to 100% | QS to 100% | QS to 100% |
| (QS to 100%) | | | | | | |
| Friction Coefficient | 0.67 | 0.65 | 0.68 | 0.69 | 0.68 | 0.70 |
| Provided | | | | | | |

(1) Polymer LR400M available from Amerchol [charge density = 0.7 meq/g, molecular weight ~400,000]
(2) Polymer LR30M available from Amerchol [charge density = 0.7 meq/g, molecular weight ~1,800,000]
(3) N-Hance 3270 available from Hercules (Aqualon), [charge density = 0.7 meq/g, molecular weight ~500,000]
(4) Viscasil 330M (General Electric Silicones)

Fluffy Coat Examples

| EXAMPLE COMPOSITION | 12 | 13 | 14 | 15 | 16 |
|---|---|---|---|---|---|
| Ammonium Laureth Sulfate | 9.00 | 10.00 | 8.00 | 8.00 | |
| (AE₃S) | | | | | |
| Ammonium Lauryl Sulfate | 7.00 | 6.00 | 8.00 | 8.00 | |
| (ALS) | | | | | |
| Sodium Laureth Sulfate | | | | | 6.00 |
| (SLE₃S) | | | | | |
| Sodium Lauryl Sulfate | | | | | 10.00 |
| (SLS) | | | | | |
| Cocamide MEA | 0.80 | 0.80 | 0.80 | 0.80 | 0.80 |
| Cetyl Alcohol | 0.90 | 0.90 | 0.90 | 0.90 | 0.90 |
| Polyquaternium-10 (1) | | | 0.20 | | |
| Guar Hydroxypropyltrimonium | 0.13 | 0.13 | | 0.13 | |
| Chloride (3) | | | | | |
| Dimethicone (4) | 1.50 | 1.35 | 1.35 | 1.50 | 1.50 |
| PEG 7M | 0.10 | 0.10 | 0.10 | 0.10 | 0.10 |
| Ethylene Glycol Distearate | 1.50 | 1.50 | 1.50 | 1.50 | 1.50 |
| Fragrance | 0.70 | 0.70 | 0.70 | 0.70 | 0.70 |
| Sodium Chloride | 0.50 | 0.50 | 0.50 | 0.50 | 0.50 |
| Citric Acid | 0.04 | 0.04 | 0.04 | 0.04 | 0.04 |
| Sodium Citrate | 0.4 | 0.4 | 0.4 | 0.4 | 0.4 |
| Sodium Benzoate | 0.25 | 0.25 | 0.25 | 0.25 | 0.25 |
| Ethylene Diamine Tetra | 0.10 | 0.10 | 0.10 | 0.10 | 0.10 |
| Acetic Acid | | | | | |
| Water and Minors | QS to 100% | QS to 100% | QS to 100% | QS to 100% | QS to 100% |
| (QS to 100%) | | | | | |
| Friction Coefficient | 0.72 | 0.73 | 0.75 | 0.72 | 0.71 |
| Provided | | | | | |

(1) Polymer LR400M available from Amerchol [charge density = 0.7 meq/g, molecular weight ~400,000]
(2) Polymer LR30M available from Amerchol [charge density = 0.7 meq/g, molecular weight ~1,800,000]
(3) N-Hance 3270 available from Hercules (Aqualon), [charge density = 0.7 meq/g, molecular weight ~500,000]
(4) Viscasil 330M (General Electric Silicones)

Wiry Coat Examples

| EXAMPLE COMPOSITION | 17 | 18 | 19 | 20 |
|---|---|---|---|---|
| Ammonium Laureth Sulfate | 10.00 | | 8.00 | 9.00 |
| (AE₃S) | | | | |
| Ammonium Lauryl Sulfate | 6.00 | | 8.00 | 7.00 |
| (ALS) | | | | |
| Sodium Laureth Sulfate | | 8.00 | | |
| (SLE₃S) | | | | |
| Sodium Lauryl Sulfate (SLS) | | 8.00 | | |
| Cocamide MEA | 0.80 | 0.80 | 0.80 | 0.80 |
| Cetyl Alcohol | 0.90 | 0.90 | 0.90 | 0.90 |
| Polyquaternium-10 (1) | | | 0.20 | |
| Guar | 0.13 | 0.13 | | 0.13 |
| Hydroxypropyltrimonium | | | | |
| Chloride (3) | | | | |
| Dimethicone (4) | 1.50 | 1.50 | 1.35 | 1.50 |
| PEG 7M | 0.10 | 0.10 | 0.10 | 0.10 |
| Ethylene Glycol Distearate | 1.50 | 1.50 | 1.50 | 1.50 |
| Fragrance | 0.70 | 0.70 | 0.70 | 0.70 |
| Sodium Chloride | 0.50 | 0.50 | 0.50 | 0.50 |
| Citric Acid | 0.04 | 0.04 | 0.04 | 0.04 |
| Sodium Citrate | 0.40 | 0.40 | 0.40 | 0.40 |
| Sodium Benzoate | 0.25 | 0.25 | 0.25 | 0.25 |
| Ethylene Diamine Tetra | 0.10 | 0.10 | 0.10 | 0.10 |
| Acetic Acid | | | | |
| Water and Minors (QS to 100%) | QS to | QS to | QS to | QS to |
| | 100% | 100% | 100% | 100% |
| Friction Coefficient Provided | 0.72 | 0.71 | 0.75 | 0.72 |

(1) Polymer LR400M available from Amerchol [charge density = 0.7 meq/g, molecular weight ~400,000]
(2) Polymer LR30M available from Amerchol [charge density = 0.7 meq/g, molecular weight ~1,800,000]
(3) N-Hance 3270 available from Hercules (Aqualon), [charge density = 0.7 meq/g, molecular weight ~500,000]
(4) Viscasil 330M (General Electric Silicones)

General Examples

| EXAMPLE COMPOSITION # | 21 | 22 | 23 | 24 | 25 | 26 |
|---|---|---|---|---|---|---|
| Ammonium Laureth Sulfate | 8.00 | 7.00 | 6.00 | | 9.00 | 7.00 |
| (AE₃S) | | | | | | |
| Ammonium Lauryl Sulfate | 8.00 | 9.00 | 10.00 | | 7.00 | 7.00 |
| (ALS) | | | | | | |
| Sodium Laureth Sulfate | | | | 8.00 | | |
| (SLE₃S) | | | | | | |
| Sodium Lauryl Sulfate | | | | 8.00 | | |
| (SLS) | | | | | | |
| Cocamidopropyl Betaine | | | | | | 2.00 |
| Cocamide MEA | 0.80 | 0.80 | 0.80 | 0.80 | 0.80 | 0.80 |
| Cetyl Alcohol | 0.90 | 0.90 | 0.90 | 0.90 | 0.90 | |
| Polyquaternium-10 (1) | | | 0.20 | | | |
| Guar Hydroxypropyltrimonium | 0.13 | | | 0.13 | | |
| Chloride (3) | | | | | | |
| Dimethicone (4) | 1.50 | 1.35 | 1.35 | 1.50 | 3.00 | 1.20 |
| PEG 7M | 0.10 | 0.10 | 0.10 | 0.10 | 0.10 | 0.10 |
| Ethylene Glycol Distearate | 1.50 | 1.50 | 1.50 | 1.50 | 1.50 | 1.50 |
| Fragrance | 0.70 | 0.70 | 0.70 | 0.70 | 0.70 | 0.70 |
| Sodium Chloride | 0.50 | 0.50 | 0.50 | 0.50 | 0.50 | 0.50 |
| Citric Acid | 0.04 | 0.04 | 0.04 | 0.04 | 0.04 | 0.04 |
| Sodium Citrate | 0.4 | 0.4 | 0.4 | 0.4 | 0.4 | 0.4 |
| Sodium Benzoate | 0.25 | 0.25 | 0.25 | 0.25 | 0.25 | 0.25 |
| Ethylene Diamine Tetra | 0.10 | 0.10 | 0.10 | 0.10 | 0.10 | 0.10 |
| Acetic Acid | | | | | | |
| Water and Minors | QS to 100% | QS to 100% | QS to 100% | QS to 100% | QS to 100% | QS to 100% |
| (QS to 100%) | | | | | | |

(1) Polymer LR400M available from Amerchol [charge density = 0.7 meq/g, molecular weight ~400,000]
(2) Polymer LR30M available from Amerchol [charge density = 0.7 meq/g, molecular weight ~1,800,000]
(3) N-Hance 3270 available from Hercules (Aqualon), [charge density = 0.7 meq/g, molecular weight ~500,000]
(4) Viscasil 330M (General Electric Silicones)

### Methods of Using

The present invention also includes methods of cleansing, conditioning and optionally therapeutically treating skin, hair and other keratinous surfaces of short haired/coated, long haired/coated, fluffy haired/coated and/or wiry haired/coated companion animals.

A method of the present invention for cleansing, conditioning, and optionally therapeutically treating skin, hair, and other keratinous surfaces of a short haired companion animal comprises the steps of:
a) contacting the skin, hair, and other keratinous surfaces of a short haired companion animal with a companion animal care composition; the composition comprising:
   i) a cleansing component; and
   ii) a conditioning component; and
b) optionally rinsing the skin, hair, and other keratinous surfaces of the short haired companion animal with water; and wherein the composition provides a friction coefficient of from about 0.75 to about 0.8.

The methods can also include the step of drying the animal, which can be done in numerous ways, as would be understood by those of skill in the art, including air drying, towel drying and/or blow drying.

When used with leave-on compositions, the methods would not require the rinsing step, but instead would include the step of leaving the composition on the animal.

As would be understood by one of skill in the art, the methods can also include additional steps such as combing, brushing, clipping, and/or other grooming of the animal during and/or after the rinsing and/or drying steps.

A method of the present invention for cleansing, conditioning, and optionally therapeutically treating skin, hair, and other keratinous surfaces of a long haired companion animal comprise the steps of:
a) contacting the skin, hair, and other keratinous surfaces of a long haired companion animal with a companion animal care composition; the composition comprising:
   iii) a cleansing component; and
   iv) a conditioning component; and
b) optionally rinsing the skin, hair, and other keratinous surfaces of the long haired companion animal with water; and wherein the composition provides a friction coefficient of from about 0.65 to about 0.75.

A method of the present invention for cleansing, conditioning, and optionally therapeutically treating skin, hair, and other keratinous surfaces of a fluffy haired and/or a wiry haired companion animal comprises the steps of:
b) contacting the skin, hair, and other keratinous surfaces of a fluffy haired and/or a wiry haired companion animal with a companion animal care composition; the composition comprising:
   i) a cleansing component; and
   ii) a conditioning component; and
b) optionally rinsing the skin, hair, and other keratinous surfaces of the fluffy haired and/or wiry haired companion animal with water; and wherein the composition provides a friction coefficient of from about 0.7 to about 0.75.

As with the methods for short haired companion animals, the methods for long haired, fluffy haired and/or wiry haired companions animals can also optionally include the step of drying the animal, which can be done in numerous ways, as would be understood by those of skill in the art, including air drying, towel drying and/or blow drying.

When used with leave-on compositions, the methods would not require the rinsing step, but instead would include the step of leaving the composition on the animal.

As would be understood by one of skill in the art, the methods can also include additional steps such as combing, brushing, clipping, and/or other grooming of the animal during and/or after the rinsing and/or drying steps.

### Articles of Commerce

The present invention also includes articles of commerce comprising a container comprising a companion animal care composition which provides cleansing, conditioning, and optionally treatment benefits to a companion animal having a short, long, fluffy and/or wiry coat, when applied, and which comprises:
a cleansing component; and
a conditioning component;
   wherein the composition provides a friction coefficient of from about 0.65 to about 0.8; and wherein the container has instructions for cleansing, conditioning, and optionally treating the companion animal. The instructions include instructions to wash as normal, massage the companion animal, optionally repeat washing, optionally dry the companion animal, and optionally groom the companion animal. The instructions can be text written in one or more languages, and/or can be and/or include pictorial representations, flow charts and diagrams.

The container can comprise any container suitable for containing compositions such as those described above, and can have any suitable cap and/or dispensing means. Non-limiting examples of such containers include containers that open at the top and must be inverted to dispense the contents; containers that open at the bottom so no inversion of the container is required to dispense the contents; spray devices having trigger and/or pump dispensing means; and combinations thereof. Non-limiting examples of a cap device include “pop” type caps that can be opened with a single hand either from above to toggle the cap open, or from below to push the cap upwards; screw/threaded type caps; and combinations thereof. Such containers, caps, and/or dispensing devices are commercially available and would be understood by one of skill in the art.

### Systems

The present invention also includes a system comprising an array of companion animal care compositions formed of a plurality of companion animal care compositions, each composition tailored for use with a particular coat type. The compositions can be marketed as an array, with accompanying indicia to enable a user to select the appropriate composition. Each composition can also include instructions for use of the composition, and indicia relating to use of the composition and the coat type for which the composition is useful.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of cleansing, conditioning, and optionally therapeutically treating skin, hair, and other keratinous surfaces of a short haired companion animal comprising the steps of:
a) contacting the skin, hair, and other keratinous surfaces of a short haired companion animal with a companion animal care composition; said composition comprising:
i) cleansing component; and
ii) a conditioning component; and
b) wherein after use of said composition, said composition provides a friction coefficient of from about 0.75 to about 0.8.

2. The method of claim 1 further comprising providing instructions for use of said companion animal care composition in said method.

3. The method of claim 1, comprising from about 0.5% to about 95%, by weight of said composition, of said cleansing component.

4. The method of claim 1, wherein said cleansing component is selected from the group consisting of anionic surfactant, non-ionic surfactant, zwitterionic surfactant, cationic surfactant, amphoteric surfactant, and mixtures thereof.

5. The method of claim 1, further comprising a treatment agent selected from the group consisting of vitamins, peptides, desquamation actives, anti-atrophy actives, anti-oxidants, flavanoids, anti-inflammatory agents, topical anesthetics, chelators, antimicrobial actives, anti-fungal actives, soothing actives, healing actives, moisturizing actives, flea actives, tick actives, other insect actives, fragrances, and mixtures thereof.

6. A method of cleansing, conditioning, and optionally therapeutically treating skin, hair, and other keratinous surfaces of a long haired companion animal comprising the steps of:
a) contacting the skin, hair, and other keratinous surfaces of a long haired companion animal with a companion animal care composition; said composition comprising:
i) a cleansing component; and
ii) a conditioning component; and
b) wherein after use of said composition, said composition provides a friction coefficient of from about 0.65 to about 0.75.

7. The method of claim 6 further comprising providing instructions for use of said companion animal care composition in said method.

8. The method of claim 6, comprising from about 0.5% to about 95%, by weight of said composition, of said cleansing component.

9. The method of claim 6, wherein said cleansing component is selected from the group consisting of anionic surfactant, non-ionic surfactant, zwitterionic surfactant, cationic surfactant, amphoteric surfactant, and mixtures thereof.

10. The method of claim 6, further comprising a treatment agent selected from the group consisting of vitamins, peptides, desquamation actives, anti-atrophy actives, anti-oxidants, flavanoids, anti-inflammatory agents, topical anesthetics, chelators, antimicrobial actives, anti-fungal actives, soothing actives, healing actives, moisturizing actives, flea actives, tick actives, other insect actives, fragrances, and mixtures thereof.

11. A method of cleansing, conditioning, or therapeutically treating skin, hair, and other keratinous surfaces of a fluffy haired or wiry haired companion animal comprising the steps of:
a) contacting the skin, hair, and other keratinous surfaces of a fluffy haired companion animal with a companion animal care composition; said composition comprising:
i) a cleansing component; and
ii) a conditioning component; and
b) wherein after use of said composition, said composition provides a friction coefficient of from about 0.7 to about 0.75.

12. The method of claim 11 further comprising providing instructions for use of said companion animal care composition in said method.

13. The method of claim 11, comprising from about 0.5% to about 95%, by weight of said composition, of said cleansing component.

14. The method of claim 11, wherein said cleansing component is selected from the group consisting of anionic surfactant, non-ionic surfactant, zwitterionic surfactant, cationic surfactant, amphoteric surfactant, and mixtures thereof.

15. The method of claim 11, further comprising a treatment agent selected from the group consisting of vitamins, peptides, desquamation actives, anti-atrophy actives, anti-oxidants, flavanoids, anti-inflammatory agents, topical anesthetics, chelators, antimicrobial actives, anti-fungal actives, soothing actives, healing actives, moisturizing actives, flea actives, tick actives, other insect actives, fragrances, and mixtures thereof.

16. An article of commerce comprising: a container comprising; a companion animal care composition, which provides cleansing, conditioning, and optionally treatment benefits to a companion animal when applied and comprises:
a) a cleansing component; and
b) a conditioning component;
wherein after use of said composition, said composition provides a friction coefficient of from about 0.65 to about 0.8; and
wherein said container comprises instructions for cleansing, conditioning, and optionally treating said companion animal, comprising instructions to wash as normal, massage said companion animal, and optionally dry said companion animal.

17. A method of cleansing, conditioning, and optionally therapeutically treating skin, hair, and other keratinous surfaces of a companion animal comprising the steps of:
a) contacting the skin, hair, and other keratinous surfaces of a companion animal with a companion animal care composition; said composition comprising:
i) a cleansing component; and
ii) a conditioning component; and
b) rinsing the skin, hair, and other keratinous surfaces of the companion animal with water; wherein after use of said composition, said composition provides a friction coefficient of from about 0.65 to about 0.8.

18. The method of claim 17 wherein said companion animal care composition further comprises a treatment agent.

19. A method of cleansing, conditioning, and optionally therapeutically treating skin, hair, and other keratinous surfaces of a companion animal comprising the steps of:
a) contacting the skin, hair, and other keratinous surfaces of a companion animal with a companion animal care composition; said composition comprising:
i) a cleansing component; and
ii) a conditioning component; and
b) leaving said composition on the skin, hair, and other keratinous surfaces of the companion animal; wherein said after use of said composition, said composition provides a friction coefficient of from about 0.65 to about 0.8.

20. The method of claim 19 wherein said companion animal care composition further comprises a treatment agent.

21. A companion animal care composition comprising: a cleansing component; and a conditioning component; wherein after use of said composition, said composition provides a friction coefficient of from about 0.65 to about 0.8.

22. The companion animal care composition of claim 21, wherein after use of said composition, said composition provides a friction coefficient of from about 0.75 to about 0.8.

23. The companion animal care composition of claim 21, wherein after use of said composition, said composition provides a friction coefficient of from about 0.65 to about 0.75.

24. The companion animal care composition of claim 21, wherein after use of said composition, said composition provides a friction coefficient of from about 0.7 to about 0.75.

25. The companion animal care composition of claim 21, comprising from about 0.5% to about 95%, by weight of said composition, of said cleansing component.

26. The companion animal care composition of claim 21, wherein said cleansing component is selected from the group consisting of anionic surfactant, non-ionic surfactant, amphoteric surfactant, zwitterionic surfactant, cationic surfactant, and mixtures thereof.

27. The companion animal care composition of claim 26, wherein said anionic surfactant is selected from the group consisting of sarcosinates, sulfates, sulfonates, isethionates, phosphates, taurates, lactylates, glutamates, carboxylates, and mixtures thereof.

28. The companion animal care composition of claim 26, wherein said non-ionic surfactant is selected from the group consisting of amine oxides, alkyl glucosides, alkyl polyglucosides, polyhydroxy fatty acid amides, alkoxylated fatty acid esters, sugar ester, phosphate esters, ethoxylated esters, glycerol esters, ethoxykates, glycerides, polyethylene glycol 20 sorbitan monolaurate (Polysorbate 20), polyethylene glycol 5 soya sterol, Steareth-20, Ceteareth-20, PPG-2 methyl glucose ether distearate, Ceteth-10, Polysorbate 80, cetyl phosphate, potassium cetyl phosphate, diethanolamine cetyl phosphate, Polysorbate 60, glyceryl stearate, PEG-100 stearate, polyoxyethylene 20 sorbitan trioleate (Polysorbate 85), sorbitan monolaurate, polyoxyethylene 4 lauryl ether sodium stearate, polyglyceryl-4 isostearate, hexyl laurate, diethanolamine cetyl phosphate, glyceryl stearate, and mixtures thereof.

29. The companion animal care composition of claim 26, wherein said zwitterionic and amphoteric surfactants are selected from the group consisting of betaines, sultaines, hydroxysultains, alkyliminoacetates, iminodialkanoates, aminoalkanoates, and mixtures thereof.

30. The companion animal care composition of claim 21, wherein said conditioning component is selected from the group consisting of silicone polymers, fatty acids, esters of fatty acids, fatty alcohols, ethoxylated alcohols, polyol polyesters, glycerin, glycerin mono-esters, glycerine polyesters, epidermal and sebaceous hydrocarbons, lanolin, straight and branched hydrocarbons, silicone oils, silicone gum, vegetable oil, vegetable oil adduct, hydrogenated vegetable oils, non-ionic polymers, natural waxes, petrolatum, petrolatum derivatives, synthetic waxes, polyolefinic glycols, polyolefinic mono-esters, polyolefinic polyesters, cholesterols, cholesterol esters, and mixtures thereof.

31. The companion animal care composition of claim 21, further comprising a treatment agent selected from the group consisting of vitamins, peptides, desquamation actives, anti-atrophy actives, anti-oxidants, flavanoids, anti-inflammatory agents, topical anesthetics, chelators, antimicrobial actives, anti-fungal actives, soothing actives, healing actives, moisturizing actives, flea actives, tick actives, other insect actives, fragrances, and mixtures thereof.

32. A companion animal care system comprising:
a) an array of companion animal care compositions, each said companion animal care composition comprising a cleansing component and a conditioning component, wherein each said companion animal care composition is tailored to a coat type of a companion animal.
b) indicia corresponding to each said companion animal care composition to enable a user to select an appropriate said companion animal care composition for a said companion animal.

33. The companion animal care system of claim 32 further comprising usage instructions for use of each said companion animal care composition.

34. The companion animal care system of claim 32 wherein said array of companion animal care compositions provides a friction coefficient of from about 0.65 to about 0.8.

35. The companion animal care system of claim 32 wherein said array comprises companion animal care compositions for use on coat types selected from the group consisting of short, long, fluffy and wiry.
